(19) United States
(12) Patent Application Publication
(10) Pub. No.: US 2023/0301754 A1
(43) Pub. Date: Sep. 28, 2023

(54) ABUTMENT FOR IMPLANT WITH CONNECTOR REDUCING DIAMETER OF SCREW HOLE

(71) Applicant: Cheon Seok JANG, Ansan-si, Gyeonggi-do (KR)

(72) Inventor: Cheon Seok JANG, Ansan-si, Gyeonggi-do (KR)

(21) Appl. No.: 18/012,940

(22) PCT Filed: Apr. 8, 2022
(86) PCT No.: PCT/KR2022/005104

§ 371 Date: Dec. 25, 2022

(30) Foreign Application Priority Data
Jul. 26, 2021 (KR) 10-2021-0097988

(51) Int. Cl. A61C 8/00

(52) U.S. Cl. CPC A61C 8/0051; A61C 8/0066; A61C 8/0068

(57) ABSTRACT

An abutment for implant including a connect reducing a diameter of a screw hole according to the present invention includes: a base provided with a coupling hole having a screw thread penetrating therethrough in a height direction of a central portion; a connector having a screw thread formed on an outer circumferential surface thereof to be threaded with the coupling hole of the base and having a screw hole penetrating along a height direction of a central portion; and a screw inserted into the screw hole to be threaded with a coupling hole and a fixture.

### [TECHNICAL FIELD

The present invention relates to an abutment for implant including a connector reducing a diameter of a screw hole, and more particularly, to an abutment for implant that is configured to have a prefabricated structure of a connector coupling an abutment to a base and an upper portion of the base and can enhance the overall aesthetics of an implant by reducing a diameter of a screw hole formed on an upper surface thereof.

### [BACKGROUND ART

An implant is made of a strong and biocompatible material such as titanium, and is a treatment method of restoring a lost tooth by implanting the material in a gum bone of a portion of the lost tooth and installing an artificial tooth on the material. The implant is a treatment method and structure capable of restoring a form and function similar to natural teeth.

In general, the implant may be configured to have an artificial tooth, an abutment, and a fixture. The fixture is a part inserted into and fixed to a gum bone, and the abutment is a part connecting the artificial tooth and the fixture.

FIG. 1 is a conceptual diagram illustrating a schematic structure of the known abutment.

Referring to FIG. 1, it can be seen that a general abutment is composed of a one-piece type in which a base serving as a body and a screw for coupling with a fixture are integrally formed, and a two-piece type in which the base and the screw are separated.

Here, the two-piece type is also called a dual abutment. Unlike the one-piece type in which the diameter of the screw hole may be adjusted, the dual abutment requires a screw to be inserted directly into the screw hole that penetrates from an upper surface to a bottom surface thereof.

In this case, generally, since the screw is configured to have a head with a larger diameter than a body at an upper portion of the body where a screw thread is formed, to accommodate this screw, a screw hole with a larger diameter than a diameter of the head of the screw may be inevitably fabricated on an upper surface of the dual abutment.

In this way, when the diameter of the screw hole increases, the diameter of the head of the screw naturally increases, and as the diameter of the head increases, even if a hole (a hole in which a coupling tool such as a driver should be inserted to fix the screw to the fixture) formed on an occlusal surface of the artificial tooth is filled with a filling material such as resin, compared to an abutment (mainly one-piece type) with a small occlusal surface or hole formed thereon, aesthetics is inevitably inferior. The problem of the phenomenon becomes evident in implants with small occlusal surfaces, such as premolars.

Referring to the related art, Korean Patent No. 1899284 discloses an abutment for implant in which a compression socket is configured to press a bolt inserted into a first insertion hole of the abutment, and as a pressure force of the compression socket continues to act on the bolt, even if vibration or the like due to self-movement continues to act on the abutment, so any loosening of the bolt may be prevented by the pressure force of the compression socket.

That is, the related art presents a structure in which the compression socket is coupled to the upper portion of the base (abutment). Although the compression socket may be possible to prevent the bolt from loosening by pressing the bolt (screw) from the upper portion, as described above, the compression socket does not play a role of reducing the diameter of the bolt (screw) itself, so the problem of having a screw hole with a large diameter still remains.

Therefore, there is a need to develop an improved structure in which a medium capable of reducing a diameter of a screw hole while reducing a height of a base in an abutment, in particular, a dual abutment is prefabricated with the base.

### DISCLOSURE

### Technical Problem

The present invention provides an abutment in which a connector is coupled to an upper portion of a base, a screw is coupled to a screw hole of the connector to reduce a diameter of the screw hole of the connector, thereby improving aesthetics while improving structural stability.

Another object of the present invention is to improve aesthetics while improving overall integrity of an abutment by configuring a connector with a head and a body and then tapering an outer circumferential surface of the head at an inclination angle corresponding to an outer circumferential surface of a base.

Still another object of the present invention is to efficiently control an external force by forming a slit on an upper surface of a connector.

Yet another object of the present invention is to strengthen a bonding force with an artificial tooth by adding a cap coupled to an upper portion of a connector.

### Technical Solution

According to the present invention, an abutment for implant including a connect reducing a diameter of a screw hole includes: a base provided with a coupling hole having a screw thread penetrating therethrough in a height direction of a central portion; a connector having a screw thread formed on an outer circumferential surface thereof to be threaded with the coupling hole of the base and having a screw hole penetrating along a height direction of a central portion; and a screw inserted into the screw hole to be threaded with a coupling hole and a fixture.

In addition, the coupling hole of the base may include a first hole threaded with at least a portion of the connector and penetrating with a first diameter, and a second hole threaded with the screw and penetrating with a second diameter smaller than the first diameter.

In addition, the base may include a lower coupling portion that is coupled to a fixture and an upper coupling portion that is located in the lower coupling portion to be coupled to an artificial tooth and is tapered to be narrow at an upper portion and wide at a lower portion at a lower height than that of the lower coupling portion, and the connector may include a body that has a screw thread coupled to the coupling hole formed on an outer circumferential surface and a head that is located in an upper portion of the body and exposed to an upper portion of the base when coupled to the base.

### [Advantageous Effects

According to an abutment for implant including a connector reducing a diameter of a screw hole according to the present invention,

1) it is possible to provide convenience in manufacturing by manufacturing the abutment in a prefabricated structure of a base and a connector, and at the same time, reduce a diameter of a hole to which a screw is coupled while the connector is coupled to the base to improve the overall aesthetics of an implant,

2) it is possible to pursue integral harmony with the base by configuring the connector with a head and a body,

3) it is possible to effectively regulate and control an external force using a slit formed recessed on an upper surface of the connector as a medium, and

4) it is possible to provide a strong coupling relationship with artificial teeth by including a cap coupled to an upper portion of the connector.

### BRIEF DESCRIPTION OF THE DRAWINGS

FIG. 1 is a conceptual diagram illustrating a known abutment.

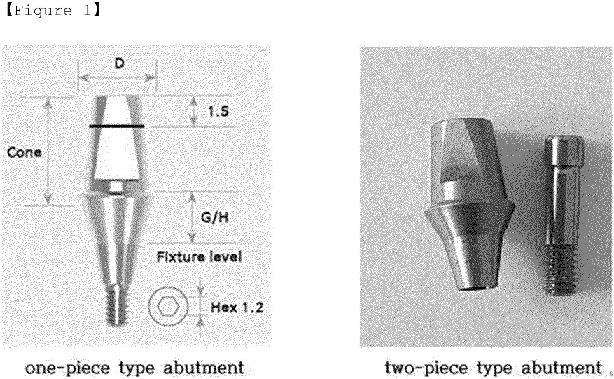

FIG. 2 is a conceptual diagram illustrating an abutment including a base and a connector of the present invention.

FIG. 3 is a cross-sectional view illustrating a structure in which a base and a screw are coupled while the connector of the present invention is configured to have a head and a body.

FIG. 4 is a cross-sectional view and a partially enlarged view illustrating a structure in which a slit is formed in the connector of the present invention.

FIG. 5 is a conceptual view illustrating a structure in which a reinforcement film is coupled to the slit of FIG. 4.

FIG. 6 is a cross-sectional view illustrating a structure in which the abutment of the present invention further includes a cap.

### BEST MODE

Hereinafter, exemplary embodiments of the present invention will be described with reference to the accompanying drawings. The accompanying drawings are not drawn to scale, and like reference numbers in each drawing indicate like elements.

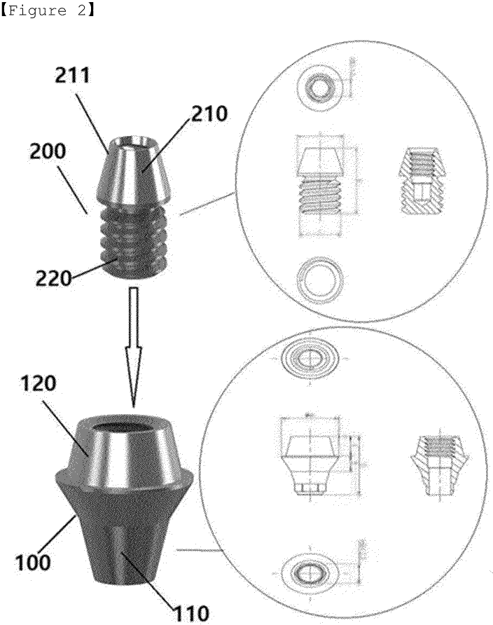

FIG. 2 is a conceptual diagram illustrating an abutment including a base and a connector of the present invention.

As can be seen from FIG. 2, an abutment 10 of the present invention is based on a structure including a base 100 and a connector 200.

The base 100 of the present invention has a structure and shape similar to a known abutment, and includes a lower coupling portion 110 that is coupled with a fixture 2 and an upper coupling portion 120 that is located on an upper portion of the lower coupling portion 110 and coupled with an artificial tooth 1.

That is, the base 100 is similar to the known abutment having an upper/lower coupling portion and is not limited to a specific shape and structure. However, the base 100 has a lower structure than the upper coupling portion of the known abutment in consideration of a coupling structure with the connector 200 to be described later. In other words, in the base 100 coupled with the connector 200, the upper coupling portion 120 has a lower standing figure than that of the lower coupling portion 110, so it is possible to prevent the total height of the connector 200 and the upper coupling portion 120 from being unnecessarily raised when the connector 200 is coupled to be exposed to the upper portion of the upper coupling portion 120 of the base 100.

The base 100 is provided with a coupling hole 130 having a screw thread formed along a height direction of the central portion.

The coupling hole 130 is similar to a screw hole of the known abutment that provides a space into which the screw 400 is inserted in order to couple the known fixture 2 and the screw 400, but in the present invention, in particular, the coupling hole 130 has a function of providing a space in which not only the screw 400 but also the connector 200 to be described later is accommodated.

This coupling hole 130 has significance in that it may be formed regardless of whether the abutment 10 of the present invention is manufactured in a one-piece type or a two-piece type.

That is, as described above, in addition to the basic function for accommodating the connector 200, the one-piece type may provide a space into which a coupling tool such as a driver for tightening the head of the screw 400 may be inserted in order to fix the integrally installed screw 400 to the fixture, and the two-piece type may provide a space into which the screw 400 as well as the above-described coupling tool may be inserted.

In this case, in the one-piece type abutment, the coupling hole 130 is recessed to a certain depth from the upper portion to the lower portion along the height direction of the central portion of the base 100, and in the two-piece type, the coupling hole 130 can be formed to penetrate along the height direction of the central portion of the base 100. That is, the coupling hole 130 of the present invention may include both the concept of a hole formed by a recess method and a penetration method.

The connector 200 of the present invention is a structure body that is accommodated in the coupling hole 130 while being threaded with the above-described coupling hole 130 of the base 100 in a state in which it has a diameter corresponding to that of the coupling hole 130.

In the connector 200, a screw hole 230 is basically formed (penetrating or recessed) along the height direction of the central portion. In this case, when the cap 300 to be described later is coupled together, the screw hole 230 of the connector 200 may not be formed to penetrate, but may be recessed to a certain length from the upper portion to the lower portion along the height direction of the central portion.

As illustrated in FIG. 2, the connector 200 may have a shape of a cone or a truncated cone as a whole, but is not limited thereto, and although not illustrated in the drawings, the connector 200 can have a shape of a cylinder or a ring.

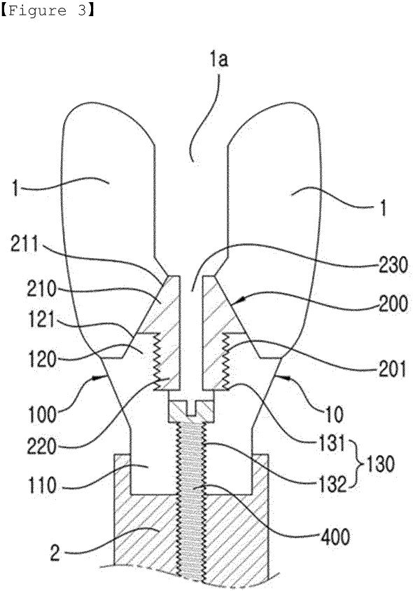

In addition, the connector 200 may be entirely accommodated in the coupling hole 130 of the base 100, but as illustrated in FIG. 3 to be described later, may have a structure in which a portion of the connector 200 is exposed to the upper portion of the base 100.

The screw hole 230 serves not only to provide a space in which a coupling tool such as a driver for fastening the screw 400 enters, but also to provide a space in which the cap 300 to be described later may be accommodated.

Specifically, when the screw hole 230 penetrates, as described above, the space into which the coupling tool enters and the space in which the cap 300 is accommodated are provided. As illustrated in the cross-sectional view of FIG. 2, when the screw hole 230 does not penetrate but is recessed, before fastening the connector 200, it is also possible to first fasten the screw 400 (apply to both single and dual types) accommodated in the coupling hole 130 to the fastener 2 via the coupling tool.

According to this structure, the diameter of the coupling hole 130, in particular, the diameter of the hole formed on the upper surface of the abutment 10 increases to ensure coupling with the screw 400 in the known abutment, resulting in poor aesthetics, thereby providing characteristics that may solve the problem of poor aesthetics.

As a specific example, the hole formed on the upper surface of the known abutment is often formed unnecessarily larger than the use for screw fastening, so the aesthetics may be poor. The connector 200 of the present invention may make the size of the hole, that is, the screw hole 230 small while adjusting the size according to various environments, so it provides a minimum space into which a coupling tool for fastening the screw 400 may enter. As a result, the connector 200 serves to enhance the natural aesthetics of the upper surface of the abutment, that is, the base 100.

As another example, when a certain period of time has elapsed after the implant is placed, there may be a problem in which the screw is loosened. It is possible to conveniently tighten the screw 400 by entering the coupling tool through the screw hole 230 of the connector 200.

In addition, when the diameter of the screw hole and screw of the known abutment is reduced, compared to the overall volume of the abutment, the volume or diameter of the screw hole becomes smaller, which may lead to a problem in that the fixture coupling force of the screw or the stable position fixing force in the coupling hole of the abutment is lowered.

That is, to solve this problem, the connector 200 of the present invention is configured to ensure the coupling force between the screw 400 and the base 100 without reducing the diameter of the coupling hole 130 of the base 100, and to faithfully perform the role of a medium capable of ensuring durability such as stiffness (i.e., non-fracture) of the base 100.

Since the base 100 and the connector 200 have an assemblable structure rather than an integral structure, compared to the integral structure made by reducing the diameter of the coupling hole 130 of the base 100, the stress or external force may be dispersed through a small gap that may occur during the assembly of the base 100 and the connector 200 to reduce the problem that the base 100 itself is fractured or broken as much as possible, and furthermore, when the connector 200 (a portion of the base occupied by the connector in the integral structure) is damaged, it may have the advantage of maintenance that only the connector 200 may be conveniently replaced without having to replace the entire implant.

In summary, compared to the case where aesthetics are enhanced by forcibly reducing the diameter of the screw hole 230 of the abutment 10 as well as the screw 400 having a head having a larger diameter than the conventionally body having a screw thread, the abutment 10 of the present invention has the connector 200, and thus, may efficiently prevent the problem of poor aesthetics due to unnecessarily large holes formed on the upper surface of the abutment 10 while ensuring structural stability and manufacturing convenience (compared to manufacturing a structure in which the connector is integrally combined with the base, manufacturing as a prefabricated method may pursue cost reduction such as mold production) of the abutment 10 as a whole.

FIG. 3 is a cross-sectional view illustrating a structure in which a base and a screw are coupled while the connector of the present invention includes a head and a body.

For example, in a two-piece type abutment, when the connector 200 is not long enough to reach the entire height of the coupling hole 130 of the base 100 (it is not necessary to manufacture the connector with a long structure corresponding to the height of the coupling hole), the connector 200 will be located in the middle or upper portion of the base 100. In this case, the screw 400 and an empty space remain in the coupling hole 130 of the remaining lower portion of the base 100. In this case, the remaining portion of the coupling hole 130 of the base 100 is configured to have a diameter for accommodating the connector 200, so, when the screw 400 having a smaller diameter than that of the connector 200 is located in the remaining portion of the coupling hole 130, the screw 400 may flow unnecessarily in the remaining portion of the coupling hole 130 due to the difference in diameter.

Of course, when the head diameter of the screw 400 is larger than that of the screw hole 230 of the connector 200, it is possible to reduce this problem. However, the screw 400 is configured to have a head and a body, but when the head of the screw 400 is larger than the diameter of the body as in the normal screw having the head/body, the above problem may remain due to the difference in diameter.

In order to prevent this problem, as can be seen from FIG. 3, the coupling hole 130 of the base 100 is preferably configured to have first and second holes 131 and 132 having diameters different from each other.

Specifically, the first hole 131 is an upper area of the coupling hole 130, and is a portion that is threaded with all or part of the connector 200 and is a portion that may accommodate the head of the screw 400. That is, the first hole 131 has a diameter corresponding to the head of the screw 400 as well as an outer diameter of the connector 200.

The second hole 132 is a lower area of the coupling hole 130, and furthermore, in a state where the screw 400 is accommodated, the second hole 132 is for the screw threaded with the body (portion where the screw thread is formed) except for the head of the screw 400, and has a second diameter smaller than the first diameter of the first hole 131, that is, a diameter corresponding to the outer diameter of the body of the screw 400.

This structure may efficiently prevent the problem in that, as described above, the structure in which the connector 200 does not have the height corresponding to the overall height of the coupling hole 130 causes the screw 400 to unnecessarily move in the lower remaining portion of the coupling hole 130 remaining after the connector 200 is coupled.

In addition, referring to FIG. 3, in the base 100 configured to have the upper coupling portion 120 and the lower coupling portion 110, it can be seen that the upper coupling portion 120 has a structure in which a taper surface 121 is tapered to be wide at the upper portion and narrow at the lower portion in a state where the lower coupling portion 110 has a low height.

This taper surface 121 is similar to the outer surface shape of the upper coupling portion of the known abutment, and is a structure formed to strengthen the coupling area with the artificial tooth or the coupling relationship with the artificial teeth stably.

As a result, the connector 200 according to FIG. 3 may be configured to have a body 220 and a head 210.

The body 220 is a portion where a screw thread is formed on the outer circumferential surface for being threaded with the coupling hole 130, and the head 210 is formed on the upper portion of the body 220. The body 220 is a portion exposed to the upper portion of the base 100 when the connector 200 is coupled with the coupling hole 130 of the base 100.

In particular, since the head 210 has a larger diameter than the body 220, the connector 200 may take a shape similar to an arrow as a whole.

That is, as illustrated in FIG. 3, the head 210 is exposed to the outside in the state in which the connector 200 includes the head 210 and the body 220. Compared to the case where the connector 200 is entirely accommodated in the coupling hole 130, it is possible to provide an advantageous characteristic of easily providing the coupling area in which the outer circumferential surface of the connector 200 may be coupled with the artificial tooth, and the height of the upper coupling portion 120 of the base 100 is reduced more than the height of the lower coupling portion 110, so for the above reasons, it is possible to pursue structural stability that allows the head 210 of the connector 200 to easily provide a coupling area with an artificial tooth while lowering the height of the connector 200 itself.

Furthermore, the outer circumferential surface of the head 210 exposed to the outside has the same or similar inclination angle as or to the tapered surface 121 of the upper coupling portion 120 on the base 100, so the head taper surface 211 that is tapered to be narrow at the upper portion and wide at the lower portion may be formed so that it may extend in a straight line without a step or a short edge (i.e., difference in an inclination angle).

Accordingly, the overall shape of the abutment 10 of the present invention may provide the advantage of providing characteristics that can ensure the integrity and stability of the area (portion of the outer circumferential surface in the upper coupling portion and the outer circumferential surface of the connector) coupled to the artificial tooth 1 while having an appearance made of a single body.

Additionally, although not illustrated in the drawings, it is possible to form a separate driver hole along the circumferential direction of the lower area of the head taper surface 211 to couple with a coupling tool such as a driver. According to this, since the driver may be operated not necessarily through the screw hole only, but also by coupling with the head taper surface 211, it is possible to provide the advantage of pursuing convenience in the assembly process.

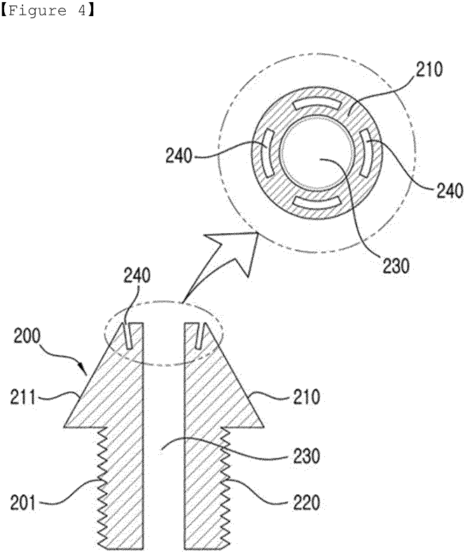

FIG. 4 is a cross-sectional view and a partially enlarged view illustrating a structure in which a slit is formed in the connector of the present invention.

As can be seen from FIG. 4, the slit 240 may be recessed in an area between the outer circumference and the portion through which the screw hole 230 penetrates on the upper surface of the connector 200.

As can be seen from the partially enlarged view of FIG. 4, a plurality of slits 240 are recessed at regular intervals along the circumferential direction of the upper surface of the connector 200, and in particular, it is not recessed vertically, but is recessed inclined toward the direction of the screw hole 230 (center direction of the upper surface of the connector).

When the slits 240 are simultaneously subjected to pressure in both directions while coupling the artificial tooth 1 to the outside and the screw 400 to the inside based on the connector 200, the slit 240 efficiently disperses the pressure force, or in a situation in which the remaining space of the screw hole 230 occurs as the screw 400 is inserted and dropped to the inside, the slit 240 serves to efficiently disperse or guide the above-described pressure force to the screw hole 400.

That is, when the head 210 of the connector 200 is firmly coupled to the artificial tooth 1, an external force is applied in a vertical direction during the coupling or occlusion of the artificial tooth 1. The slit 240 provides a characteristic capable of dispersing this external force in the extension direction of the slit 240, that is, in the inclined direction which is the screw hole 230 direction.

As a result, by efficiently inducing the phenomenon in which unnecessary pressure is applied to the fixture 2 or the alveolar bone by the vertical external force of the artificial tooth 1 toward the screw hole 230, which is the remaining space in which the screw 400 is accommodated, it is possible to ensure the overall stability of the abutment 10, and since the direction of the screw hole 230 is toward the center of the connector 200, even if there is a deviation in external force, it is possible to efficiently disperse and alleviate the deviation in external force.

In particular, such a slit 240 is more useful in a structure in which the artificial tooth 1 and the screw 400 are located inside and outside the head 210 in the connector 200 having the head 210 and are subjected to pressure.

In addition, since the connector 200 is usually made of a metal material having rigidity, bending does not occur, but due to the formation of the slit 24, a fine elastic force is provided to a portion of the head 210, so it is possible to disperse the external force applied to the connector 200 (particularly, the head) .

Furthermore, an expansion hole 241 having a larger diameter than the slit 240 may be formed at an inner end of the slit 240.

When teeth are weakly occluded and thus an occlusion force is not great or the occlusion force of the artificial tooth 1 is weak, as described above, the external force such as the occlusal force may be sufficiently dispersed only by the slit 240. However, when the relatively large occlusal force is applied, the stress is concentrated in the central portion or both ends, the fatigue of the corresponding area increases, which may lead to fracture from the corresponding portion when the abutment 10 is used for a long period of time. However, since the expansion hole 241, which has a larger diameter than that of the slit 240 and secures sufficient space, may efficiently distribute the stress concentrated on the inner end of the slit 240, even if the abutment 10 is used for a longer period of time, the stability of the implant can be ensured so that the fatigue does not relatively increase.

That is, the expansion hole 241 has an improved structure provided by maximally supplementing the disadvantages of the thin and long incised slit 240, so the expansion hole ensures the overall durability of the implant as well as the abutment 10 and serves to sufficiently strengthen the occlusal force and stress dispersion function.

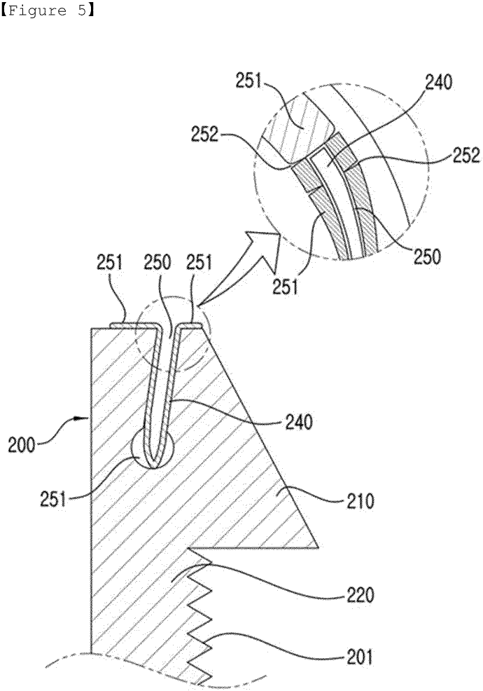

FIG. 5 is a conceptual view illustrating a structure in which a reinforcement film is coupled to the slit of FIG. 4.

As can be seen from FIG. 5, it is possible that a reinforcement film 250 is additionally fitted to the above-described slit 240 in order to enhance a buffering action against external force.

The reinforcement film 250 takes a structure in which the longitudinal section shape extends in a U shape. In other words, since the slit 240 has a three-dimensional hexahedron or cylindrical shape, the reinforcement film 250 means that the upper portion has an open reverse dome or cup shape.

The reinforcement film 250 is made of a human-friendly elastic material, for example, silicon, and more preferably, a biocompatible material such as polydionaxone (PDO), PLLA, and PCL that may be absorbed by the human body, and therefore, even if a small amount of reinforcement film 250 is escaped or separated from the slit 240, it is possible that the reinforcement film 250 may be spit out by the user or dissolved in the body unconsciously.

As described above, the reinforcement film 250 maximizes the elasticity of the reinforcement film 250 when a small amount of elasticity is applied to the surrounding area by the slit 240, and when the slit 240 shrinks or condenses in a small amount, the external force may be absorbed by the elasticity of the reinforcement film 250.

In particular, since the reinforcement film 250 is not made of a single block or film but takes a U-shaped longitudinal section shape, that is, a structure in which the inlet side of the slit 240 is open, a lower end of the reinforcement film 250 acts like a hinge, and both ends of the reinforcement film 250 may rotate in small amounts, so that the external force concentrated on the inner end (lower portion) of the slit 240 may be efficiently distributed toward the upper portion of the slit 240.

Furthermore, both ends of the reinforcement film 250 are bent and extend to the upper area of the connector 200 around the inlet of the slit 240, and an expansion part 251 can be formed between cracks 252 formed at regular intervals along the extension direction of the slit 240.

That is, as described above, since the reinforcement film 250 does not have a flat surface but has a three-dimensional shape extending along the inner circumferential surface of the slit 240, when a portion exposed to the outside of the slit 240 is simply folded, the exposed portion is not folded, For this reason, the cracks 252 are formed at regular intervals, and the fragments divided based on the cracks 252 can be folded to contact the upper surface of the connector 200, and the folded portion is called an extension portion 251. Through these cracks 252, the overall shape of the expansion part 251 may maintain a ring shape more stably, thereby ensuring a close contact relationship with the artificial tooth 1.

An upper surface of the expansion part 251 may be recessed with a certain depth and shape in order to enhance adhesion with the artificial tooth 1. That is, since the lower portion of the artificial tooth 1 has a round three-dimensional shape, the seating groove may also be round correspondingly, that is, rounded, and recessed.

In the partially enlarged view of FIG. 5, in the expansion part 251 divided into five parts based on the crack 252, a partial area or all area of the upper surface is designed according to a contact portion of the artificial tooth 1, so the shape of the seating groove may be determined to fit the shape of the artificial tooth 1.

In summary, the extension portion 251 and the seating groove formed on the upper surface thereof serve to safely support the artificial tooth 1 and ensure a close contact relationship between the artificial tooth 1 and the extension portion 251, thereby more smoothly performing the basic function of the reinforcement film 250, and the expansion portion 251 primarily absorbs and processes the coupling force or the occlusal force generated by the artificial tooth 1 to provide characteristics capable of pursuing the structural stability of the abutment 10 as well as the connector 200.

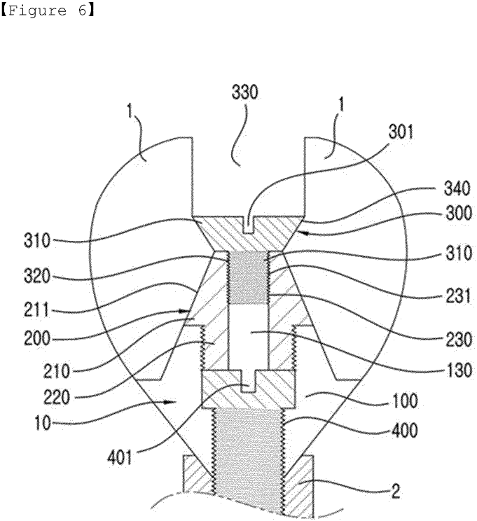

FIG. 6 is a cross-sectional view illustrating a structure in which the abutment of the present invention further includes a cap.

In the abutment 10 of the present invention illustrated in FIGS. 2 to 5, the coupling structure of the base 100 and the connector 200 has been described. Here, it is possible that the cap 300 is additionally coupled.

As can be seen from FIG. 6, the cap 300 is coupled to the upper portion of the connector 200 including the head 210 and the body 220. In this case, the upper portion or entire of the area of the screw hole 230 formed in the head 210 may be provided with the screw thread.

The cap 300 has a structure coupled to the screw hole 230 of the head 210 and exposed to the upper portion of the head 210. In this case, at least the upper area of the screw hole 230 and the outer circumferential surface of the cap 300 are provided with the screw thread, so the connector 200 can be threaded with the screw hole 230 of the head 210.

The inside of the cap 300 is sealed and may serve as a lid for sealing the upper portion of the abutment 10, in detail, the upper portion of the connector 200.

There may be various methods for mounting the connector 200 including the cap 300 on the base 100 or the like. However, after the base 100 and the fixture 2 are coupled via the screw 400, it is preferable to go through a process of accommodating the connector 200 in the coupling hole 130 of the base 100 and then accommodating and coupling the cap 300 to the screw hole 230 of the connector 200.

Like the connector 200, the cap 300 may be configured to include a cap head 310 and a cap body 320. The screw thread is provided on the outer circumferential surface of the cap body 320, and when the cap 300 is coupled to the connector 200, the cap head 310 is exposed to the upper portion of the connector 200 and serves to increase the bonding area with the artificial tooth 1.

Such a cap 300 provides the convenience of being conveniently manufactured as a prefabricated assembly when it is difficult to manufacture to increase the height of the abutment 10 (or dual abutment) composed of a single body, as well as may increase the bonding area with the artificial tooth 1 to pursue a strong and stable coupling structure between the artificial tooth 1 and the abutment 10.

Furthermore, in order to increase the coupling area with the artificial tooth 1 and prevent the artificial tooth 1 from escaping upward, as described above, in the state in which the head taper surface 211 in which the outer circumferential surface of the head 210 is tapered to be wide at the upper portion and narrow at the lower portion to correspond to the tapered surface 121 of the upper coupling portion 120 is formed, the outer circumferential surface of the cap 300 may be formed with the cap taper surface 340 that is tapered to be wide at the upper portion and narrow at the lower portion to be symmetrical (x-axis symmetry, which is an imaginary horizontal line) to the inclination angle of the head taper surface 211.

According to this structure, since the head 210 and the cap 300 of the connector 200 take a shape similar to that of a “ribbon” rotated 90°, the coupling area with the artificial tooth 1 increases as well as the problem of the artificial tooth 1 escaping in the upward direction is completely prevented due to the reverse taper structure of the cap 300 (inclination to spread upward), so it is possible to increase the occlusal force with the artificial tooth 1.

As described so far, the configuration and operation of the abutment for implant including the connector for reducing the diameter of the screw hole according to the present invention are described and illustrated in the above description and drawings, but this is only an example. Therefore, it goes without saying that the configuration and operation of the abutment for implant can be variously changed and modified without deviating from the technical spirit of the present invention.

1] An abutment for implant including a connector reducing a diameter of a screw hole, the abutment comprising:
a base provided with a coupling hole having a screw thread formed along a height direction of a central portion and including a lower coupling portion that is coupled to a fixture and an upper coupling portion that is located in the lower coupling portion to be coupled to an artificial tooth and is tapered to be narrow at an upper portion and wide at a lower portion at a lower height than that of the lower coupling portion;
a connector provided with a screw hole formed along the height direction of the central portion and including a body that has a screw thread coupled to the coupling hole formed on an outer circumferential surface and a head that is located in an upper portion of the body and exposed to an upper portion of the base when coupled to the base; and
a screw inserted into the screw hole to be threaded with the coupling hole and the fixture,
wherein the outer circumferential surface of the head is formed with a head tapered surface that is tapered to be narrow at the upper portion and wide at the lower portion to correspond to an inclination angle of the tapered surface of the upper coupling portion.

2] The abutment of claim 1, wherein the coupling hole of the base comprises: a first hole threaded with at least a portion of the connector and penetrating with a first diameter; and a second hole threaded with the screw and penetrating with a second diameter smaller than the first diameter.

3] The abutment of claim 1, wherein an area between the screw hole and an outer circumference on an upper surface of the connector comprises a slit obliquely recessed in a direction of the screw hole at regular intervals along a circumferential direction of the upper surface of the connector.

4] The abutment of claim 3, wherein an inner end of the slit is provided with an expansion hole having a larger diameter than that of the slit.

5] The abutment of claim 3, wherein a reinforcement film made of an elastic material having a U-shaped longitudinal section surface is fitted into the slit.

6] The abutment of claim 5, wherein an extension portion that is bent and extends to an upper area of the connector around an inlet of the slit and has an upper surface provided with a seating groove recessed so that an artificial tooth is seated between cracks formed at regular intervals along an extending direction of the slit.

7] The abutment of claim 1, comprising:
a cap that is coupled to the screw hole of the head and exposed to an upper portion of the head.

8] The abutment of claim 7, wherein an outer circumferential surface of the head is provided with a head taper surface tapered to be narrow at the upper portion and wide at the lower portion to correspond to the tapered surface of the upper coupling portion, and
an outer circumferential surface of the cap is provided with a cap taper surface tapered to be wide at the upper portion and narrow at the lower portion to be symmetrical to an inclination angle of the head taper surface.
